(19) United States
(12) Patent Application Publication
(10) Pub. No.: US 2013/0137737 A1
(43) Pub. Date: May 30, 2013

(54) HIGHLY CRYSTALLINE VALSARTAN

(71) Applicants: Jens Burgbacher, Basel (CH); Björn Thomas Hahn, Mannheim (DE); Florian Andreas Rampf, Hegenheim (FR); Ricardo Schneeberger, Wentzwiller (FR)

(72) Inventors: Jens Burgbacher, Basel (CH); Björn Thomas Hahn, Mannheim (DE); Florian Andreas Rampf, Hegenheim (FR); Ricardo Schneeberger, Wentzwiller (FR)

(73) Assignee: NOVARTIS AG, Basel (CH)

(21) Appl. No.: 13/813,181

(22) PCT Filed: Aug. 1, 2011
(86) PCT No.: PCT/EP2011/063254

§ 371 Date: Jan. 30, 2013

Related U.S. Application Data
(60) Provisional application No. 61/370,285, filed on Aug. 3, 2010.

(51) Int. Cl. C07D 257/04

(52) U.S. Cl. CPC C07D 257/04
USPC 514/381; 548/253

(57) ABSTRACT

The present invention describes a highly crystalline form of valsartan, pharmaceutical compositions thereof and process for the preparation thereof.

### BACKGROUND OF THE INVENTION

Polymorphs of valsartan and/or salts thereof are described in China patent publication 200410067406.8, WO2004/083192; WO2007/017897; US Patent Publication 2008/0261959; WO2003/089417 A1; WO2006/076561 A1; WO2003/066606; WO2002/06253, U.S. Pat. No. 6,869,970. However, there remains a need to provide a form of valsartan that has a greater degree of crystallinity compared to known forms or polymorphs of valsartan.

### SUMMARY OF THE INVENTION

The present invention relates to a novel, highly crystalline form of valsartan, pharmaceutical compositions thereof and process for the preparation thereof.

In one embodiment, the present invention is directed toward a highly crystalline form of valsartan characterized by an XRPD pattern with a peak at about 31.0±10.2 degrees 2-theta and substantially lacking X-ray diffraction peaks between 0 and 8±10.2 degrees 2-theta.

In another embodiment, the present invention is directed toward a highly crystalline form of valsartan having a peak melting point temperature of 140.8° C.±3° C.

In another embodiment, the present invention is directed toward a highly crystalline form of valsartan having a single crystalline structure defined by the following peak positions:

### Peak Position

[°]
9.308
11.643
13.854
16.056
17.643
18.561
19.186
20.024
20.567
21.335
24.597
25.051
26.292
31.032

In another embodiment, the present invention is directed toward a process for the preparation of a highly crystalline form of valsartan comprising:

(a) combining solid valsartan with a solvent that is an ester,

(b) heating said combination to a temperature below complete dissolution of the solid valsartan;

(c) stirring said mixture for a time effective to form a suspension with the solvents therein that form a mother liquor;

(d) separating the solids in the suspension from the mother liquor; and

(e) drying said solids to give a highly crystalline form of valsartan.

In another embodiment, the present invention is directed toward a pharmaceutical composition comprising a pharmaceutically effective amount of the highly crystalline form of valsartan in combination with a pharmaceutically acceptable carrier.

In another embodiment, the present invention is directed toward a method for treating hypertension or elevated blood pressure in a patient comprising administering a pharmaceutical composition comprising a pharmaceutically effective amount of the highly crystalline form of valsartan in combination with a pharmaceutically acceptable carrier to a patient in need thereof.

The present invention has the advantage of providing a highly crystalline form of valsartan that can be easily dried compared to known forms of valsartan.

The present invention has the advantage of providing a highly crystalline form of valsartan that has as low or even a lower residual solvent content compared to known forms of valsartan.

The present invention has the advantage of providing a highly crystalline form of valsartan that has a crystallinity close to or about 100%.

The present invention has the advantage of providing a highly crystalline form of valsartan that has a stability as high or even higher compared to known forms of valsartan.

The present invention has the advantage of providing a highly crystalline form of valsartan that has a purity as high or even higher compared to known forms of valsartan.

### BRIEF DESCRIPTION OF THE FIGURES

FIG. 1 is a picture depicting the morphology of the highly crystalline valsartan of the present invention by Scanning Electron Microscopy (SEM) at a resolution of 1 millimeter (mm) or 1000 microns or micrometers (μm)

FIG. 2 is a picture depicting the morphology of the highly crystalline valsartan of the present invention by SEM at a resolution of 200 microns or micrometers (μm)

FIG. 3 is a picture depicting the morphology of the highly crystalline valsartan of the present invention by SEM at a resolution of 50 μm

FIG. 4 is a picture depicting the morphology of the highly crystalline valsartan of the present invention by SEM at a resolution of 20 μm

FIG. 5 is a picture depicting the morphology of the highly crystalline valsartan of the present invention by SEM at a resolution of 20 μm

### DETAILED DESCRIPTION OF THE FIGURES

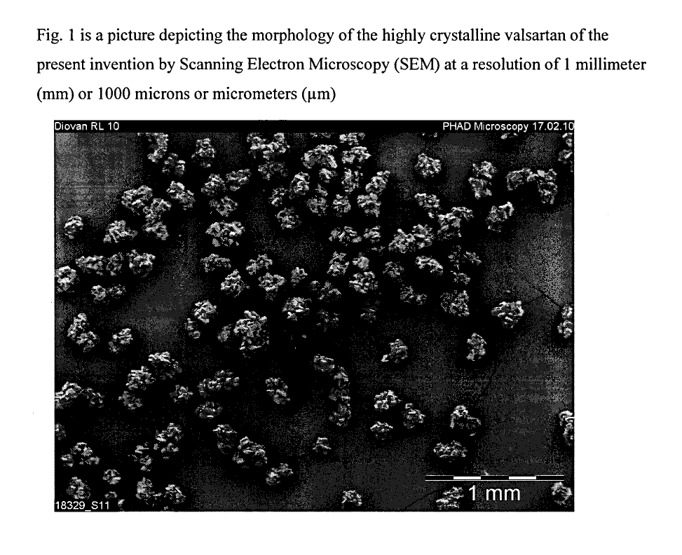

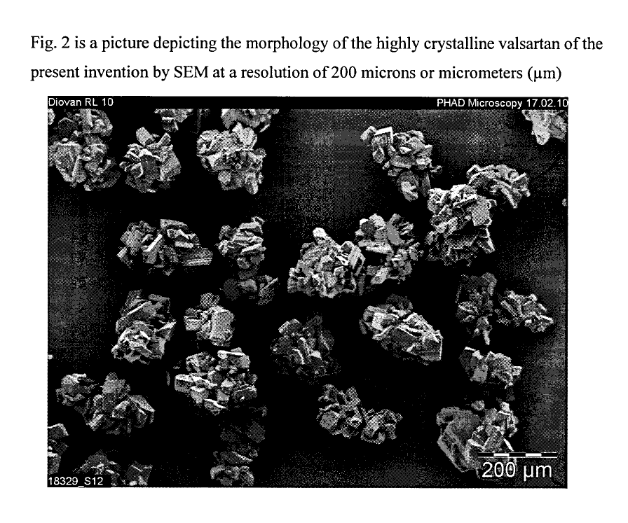

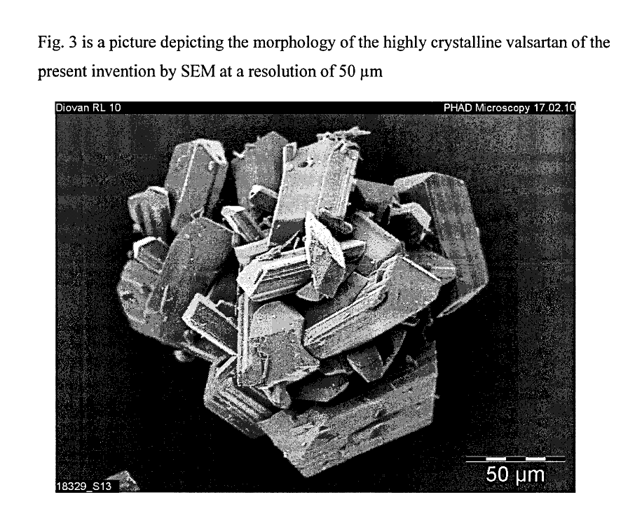

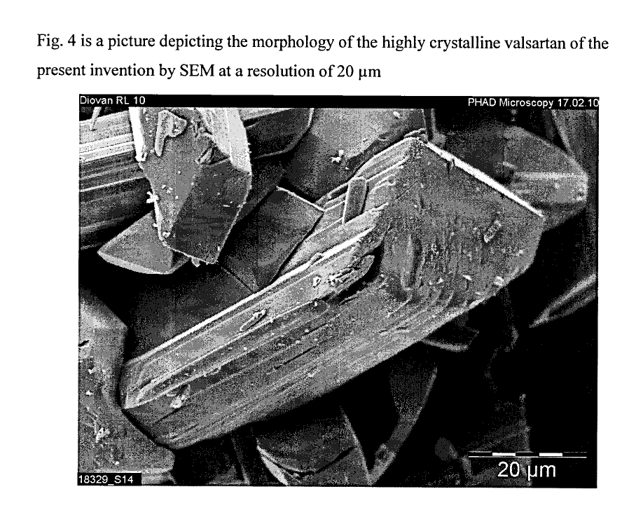

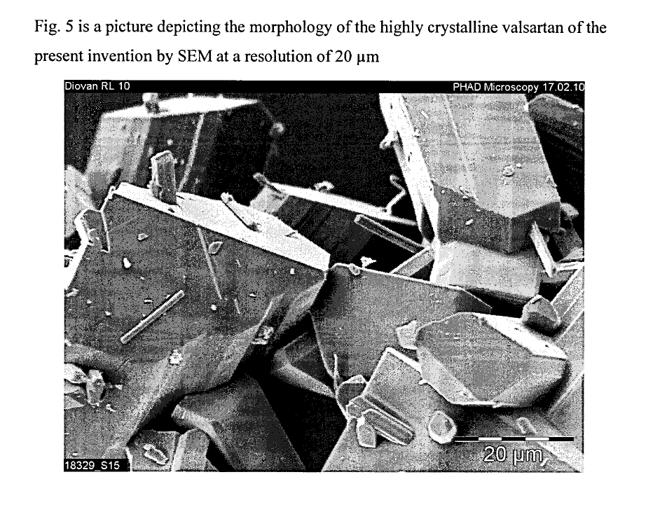

FIGS. 1-5 depict the morphology of the highly crystalline valsartan of the present invention by Scanning Electron Microscopy (SEM). In the highly crystalline valsartan, the molecules are packed in a dense 3-Dimensional solid state, as there are extremely few or no detectable channels or water molecules associated with the highly crystalline structure. The highly crystalline valsartan is also characterized as well individualised, quasi flower-like conglomerates up to ˜200 μm in diameter. The spheroid conglomerates consist of fused elongate columnar crystals of irregular tetrahedral shape factor and a length profile between ˜12 and 90 μm. The crystals exhibit well defined sharp edges, lined surfaces (likely twining planes), some incidence of fracture planes and, occasionally, pitted surfaces particularly on the crystal ends. The significant formation of the spheroid conglomerates is believed to account, in part, for the high flowability of the highly crystalline valsartan.

### DETAILED DESCRIPTION OF THE INVENTION

Valsartan has the molecular structure of which is shown below

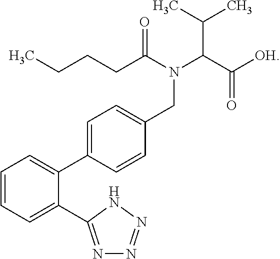 [SMILES: CCCCC(=O)N(Cc1ccc(-c2ccccc2-c2nnn[nH]2)cc1)C(C(=O)O)C(C)C]

Valsartan may be in the racemic form or as one of the two isomers shown below

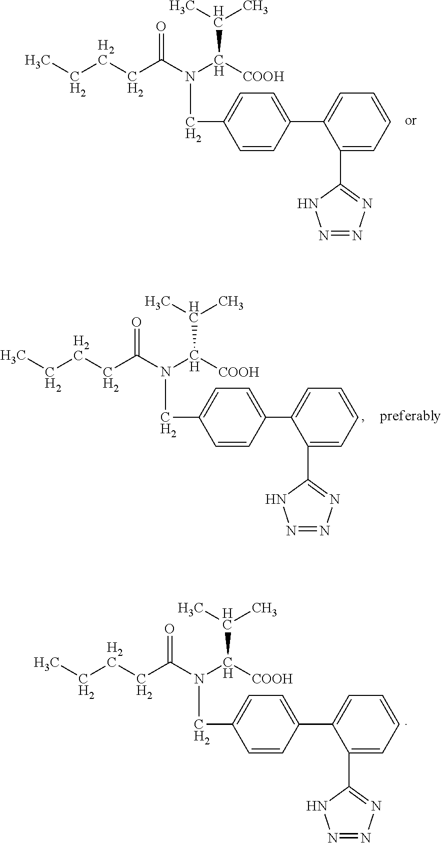 [SMILES: CCCCC(=O)N(Cc1ccc(-c2ccccc2-c2nnn[nH]2)cc1)[C@H](C(=O)O)C(C)C ; CCCCC(=O)N(Cc1ccc(-c2ccccc2-c2nnn[nH]2)cc1)[C@@H](C(=O)O)C(C)C ; CCCCC(=O)N(Cc1ccc(-c2ccccc2-c2nnn[nH]2)cc1)[C@H](C(=O)O)C(C)C]

Valsartan is known as ((S)-N-valeryl-N-{[2′-(1H-tetrazole-5-yl)-biphenyl-4-yl]-methyl}-valine) and also known as N-(1-oxopentyl)-N-[[2′-(1H-tetrazol-5-yl) [1,1′-biphenyl]-4-yl]methyl]-L-valine used according to the present invention can be purchased from commercial sources or can be prepared according to known methods. For example, the preparation of valsartan is described in U.S. Pat. No. 5,399,578 and EP 0 443 983, the disclosure of which is incorporated herein by reference. Valsartan may be used for purposes of this invention in its free acid form, as well as in any suitable salt form. The term “substantially lacking” refers to the substantial absence of any major or minor peaks in the spectrum being measured.

The present invention is directed to a process for the preparation of a highly crystalline form of valsartan comprising:

(a) combing solid valsartan with a solvent that is an ester,

(b) heating said combination to a temperature below complete dissolution of the solid valsartan;

(c) stirring said mixture for a time effective to form a suspension with the solvents therein that form a mother liquor;

(d) separating the solids in the suspension from the mother liquor; and

(e) drying said solids to give a highly crystalline form of valsartan.

In step (a) the valsartan is combined with a first solvent or organic ester such as methyl acetate, ethyl acetate, isopropyl acetate, isobutyl acetate or mixtures thereof. In one embodiment the valsartan is combined with ethyl acetate, In another embodiment, valsartan is combined with ethyl acetate and isobutylacetate. Optionally, the combination of valsartan and organic ester can also be admixed with a second solvent, such as a ketone, alcohol, aliphatic, aromatic solvent or mixtures thereof. Suitable ketone solvents include methylisobutylketone. Suitable alcohol solvents includes C-1 to C-10 alcohols, such as methanol, ethanol, n-propanol, isopropanol, n-butanol, t-butanol, pentanols, and decanol. Suitable aliphatic solvents include C-5 to C-10 alkanes such as pentane, n-hexane, cyclohexane, n-heptane, and cycloheptane. Suitable aromatic solvents include benzene and toluene. In one embodiment, the valsartan is combined with a mixture of ethyl acetate and toluene. In another embodiment, the valsartan is combined with a mixture of ethyl acetate and cyclohexane. The weight ratio of the first solvent to the second solvent can range from 100 to 1, preferably from about 20 to 30:1 (first solvent:second solvent).

In step (b) the combination of valsartan and organic solvent(s) can be heated to a temperature below complete dissolution of the solid valsartan. That is, the temperature is such to avoid or minimize complete dissolution of the solid valsartan. Such temperature can range from about 30-60 degrees Celsius (° C.), or more preferentially from about 48-50° C.

In step (c) the heated combination is stirred or agitated for a time effective to form a suspension with the solvents therein that form the mother liquor, such as the first solvent(s) and optional second solvent(s) at a temperature similar to that described in step (b). Such stirring or agitation may performed by any known means, including stirrers, sonification, tumble mixing and the like.

In step (d) the solids in the suspension are separated from the mother liquor by any known means, such as filtration, decantation, centrifugation and the like. During separation from the mother liquor, preferably the solids are maintain at a temperature approximate or similar to the temperature(s) described in step (b) above.

In step (e) the solids can be dried by any known means, such as by heating, vacuum drying, air drying, dessicants and the like to give the highly crystalline form of valsartan. Such temperature(s) can range from about 50° C. to below the melting point of valsartan.

The highly crystalline form of valsatan prepared has a crystallinity of at least 98%. Forms of even higher cystallinity can be prepared such as at least 99% or even about 100%. Such highly crystalline forms of valsartan are substantially devoid of solvents or other occluded materials.

Such highly crystalline form of valsartan has a peak melting point temperature of 140.8° C.±3° C. Methods for measuring such peak temperarture can use a heating rate of 10° C./minute with a suitable crucible or capsule for measurement, such as AL-CRUCIBLES 40 ml; ME-26763.

The highly crystalline form of valsartan can be further crystallized in other organic solvents such as ketones, esters and C1-C6 alcohols.

In another embodiment, the present invention is directed toward a pharmaceutical composition comprising a pharmaceutically effective amount of the highly crystalline form of valsartan in combination with a pharmaceutically acceptable carrier. Such carriers are described hereinbefore.

In another embodiment, the present invention is directed toward a method for treating hypertension or elevated blood pressure in a patient comprising administering a pharmaceutical composition comprising a pharmaceutically effective amount of the highly crystalline form of valsartan in combination with a pharmaceutically acceptable carrier to a patient in need thereof.

The following examples are provided to illustrate, but not limit the scope of the invention.

### EXAMPLE 1a

### Preparation of Highly Crystalline Valsartan

To a glass round-bottom flask (0.9 liters, SV01-reactor) equipped with a reactor water jacket and a 4-blade anchor stirrer, was added 96 g valsartan, 486 g ethyl acetate and 18 g toluene at room temperature. The reactor jacket temperature was heated or raised to 48-50 degrees Celsius (° C.), the stirrer was set at 100 revolutions per minute (rpm) and ingredients in the flask were stirred for at least 24 hours, forming a suspension. The heated suspension was passed through a glass vacuum filter heated to a temperature of 50° C., which separated the solids from the mother liquor. The solids were dried at 50° C. in a vacuum oven to give a highly crystalline form of valsartan.

### EXAMPLE 1b

### X-Ray Powder Diffraction Analysis of Highly Crystalline Valsartan

Analysis of the highly crystalline valsartan of Example 1 by X-Ray Powder Diffraction (XRPD) revealed the following crystallographic data. The major peaks are represented in bold.

### Valsartan Highly Crystalline Form XRPD Peaks

| Peak No.: | Peak position [°] | Accuracy [+/−°] | Peak Type |
|---|---|---|---|
| 1 | 9.308 | 0.2 | major |
| 2 | 10.74 | 0.2 | minor |
| 3 | 11.643 | 0.2 | major |
| 4 | 13.854 | 0.2 | major |
| 5 | 15.136 | 0.2 | minor |
| 6 | 16.056 | 0.2 | major |
| 7 | 16.686 | 0.2 | minor |
| 8 | 17.643 | 0.2 | major |
| 9 | 18.561 | 0.2 | major |
| 10 | 19.186 | 0.2 | major |
| 11 | 20.024 | 0.2 | major |
| 12 | 20.567 | 0.2 | major |
| 13 | 21.335 | 0.2 | major |
| 14 | 21.595 | 0.2 | minor |
| 15 | 21.858 | 0.2 | minor |
| 16 | 22.879 | 0.2 | minor |
| 17 | 24.597 | 0.2 | major |
| 18 | 25.051 | 0.2 | major |
| 19 | 26.292 | 0.2 | major |
| 20 | 31.032 | 0.2 | major |

### EXAMPLE 1c

### Three-Dimensional Crystallography of Highly Crystalline Valsartan

Additional crystallographic information on the highly crystalline valsartan form in Example 1 was obtained from a single crystal measurement and defines the crystalline structure of the larger crystalline form.

| Symmetry cell setting | Orthorhombic |
|---|---|
| Symmetry Space group name H-M | P212121 |
| Cell length a | 7.3728 (6) |
| Cell length b | 16.3876 (13) |
| Cell length c | 18.8376 (14) |
| Cell angle alpha | 90.00 |
| Cell angle beta | 90.00 |
| Cell angle gamma | 90.00 |
| Cell volume | 2276.0 (3) |
| Cell fomula units Z | 4 |
| Experimental crystal density diffrn | 1.271 |

### EXAMPLE 1d

### Morphology of the Highly Crystalline Form

Scanning Electron Microscopy (SEM) is used to show the shape or morphology of the highly crystalline form of valsartan as shown in FIGS. 1-5.

1. A highly crystalline form of valsartan characterized by an XRPD pattern with a peak at about 31.0±0.2 degrees 2-theta and substantially lacking X-ray diffraction peaks between 0 and 8±0.2 degrees 2-theta.

2. A highly crystalline form of valsartan having a peak melting point temperature of 140.8° C.±3° C.

3. A highly crystalline form of valsartan having a single crystalline structure defined by the following peak positions:
Peak position
[°]
9.308
11.643
13.854
16.056
17.643
18.561
19.186
20.024
20.567
21.335
24.597
25.051
26.292
31.032

4. A process for the preparation of a highly crystalline form of valsartan comprising:
(a) combining solid valsartan with a solvent that is an ester,
(b) heating said combination to a temperature below complete dissolution of the solid valsartan;
(c) stirring said mixture for a time effective to form a suspension with the solvents therein that form a mother liquor;
(d) separating the solids in the suspension from the mother liquor; and
(e) drying said solids to give a highly crystalline form of valsartan.

5. A pharmaceutical composition comprising a pharmaceutically effective amount of the highly crystalline form of valsartan of claim 1 in combination with a pharmaceutically acceptable carrier.

6. A method for treating hypertension or elevated blood pressure in a patient comprising administering a pharmaceutical composition comprising a pharmaceutically effective amount of the highly crystalline form of valsartan of claim 1 in combination with a pharmaceutically acceptable carrier to a patient in need thereof.
